(19) United States
(12) Patent Application Publication
(10) Pub. No.: US 2023/0086296 A1
(43) Pub. Date: Mar. 23, 2023

(54) WEARABLE DEVICE AND DETECTION METHOD

(71) Applicant: JSR CORPORATION, Tokyo (JP)

(72) Inventors: Katsutoshi Sawada, Tokyo (JP); Masayasu Fujioka, Tokyo (JP); Shun Aoki, Tokyo (JP); ShouAdnas Takahashi, Tokyo (JP); Nobutoshi Kobayashi, Tokyo (JP)

(73) Assignee: JSR CORPORATION, Tokyo (JP)

(21) Appl. No.: 18/058,783

(22) Filed: Nov. 25, 2022

Related U.S. Application Data
(63) Continuation of application No. PCT/JP2021/018663, filed on May 17, 2021.

(30) Foreign Application Priority Data
May 29, 2020 (JP) 2020-094355

(51) Int. Cl. A61B 5/259; A61B 5/28; A61B 5/332

(52) U.S. Cl. CPC A61B 5/259; A61B 5/28; A61B 5/332

(57) ABSTRACT

A wearable device according to an embodiment includes a detection unit, a first layer, a second layer, and a third layer. The detection unit detects the living body information. The first layer is flexible and disposed in a first direction relative to the detection unit and includes a conductor electrically connected to the detection unit. The second layer is disposed in the first direction relative to the first layer and is harder than the first layer. The third layer is disposed in the first direction relative to the second layer and includes an electronic component electrically connected to the conductor.

### CROSS-REFERENCE TO RELATED APPLICATION

This application is a continuation of PCT International Application No. PCT/JP2021/018663 filed on May 17, 2021 which claims the benefit of priority from Japanese Patent Application No. 2020-094355 filed on May 29, 2020, the entire contents of which are incorporated herein by reference.

### BACKGROUND OF THE INVENTION

### 1. Field of the Invention

An embodiment of the present invention relates to a wearable device and a detection method.

### 2. Description of the Related Art

Recently, various kinds of technologies of detecting living body information of a human by using a human-wearable device have been disclosed. For example, according to a disclosed technology, a small-sized and lightweight electrocardiographic sensor is bonded to skin with an adhesive tape or the like to acquire a continuous electrocardiogram. It is expected that continuous living body information in daily life is acquired by using such a wearable device. The related technology is described, for example, in Japanese Patent No. 6539827.

A wearable device potentially cannot acquire living body information due to various factors in daily life. For example, the wearable device is potentially deflected and damaged by motion of a human body. In addition, the wearable device may cause discomfort to a wearer due to sweat (water) and be separated from skin of the wearer.

### SUMMARY OF THE INVENTION

It is an object of the present invention to at least partially solve the problems in the conventional technology.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating an exemplary structure of a wearable device 100 according to an embodiment;

FIG. 2 is a diagram illustrating an exemplary structure of a flexible substrate 110 and an electronic component unit 120 according to the embodiment;

FIG. 3 is a diagram illustrating exemplary wiring on the flexible substrate 110 and a hard substrate 121 according to the embodiment;

FIG. 4 is a diagram illustrating an exemplary structure of an adhesive unit 130 according to the embodiment;

FIG. 5 is a diagram for description of a bonding method for the wearable device 100 according to the embodiment; and

FIG. 6 is a diagram illustrating an exemplary structure of the adhesive unit 130 according to a modification.

### DETAILED DESCRIPTION OF THE PREFERRED EMBODIMENTS

The following describes a wearable device and a detection method according to an embodiment with reference to the accompanying drawings. The embodiment described below is not limited to the following description. The embodiment described below may be combined with another embodiment or a conventional technology to the extent not inconsistent with any configuration. The embodiment described below may include a configuration disclosed in Japanese Patent No. 6539827.

Any configuration described below with reference to the drawings is not limited to illustrated contents. For example, the dimension and angle of each component described below with reference to the drawings may be changed as appropriate to the extent not impairing functions of the wearable device.

### EMBODIMENT

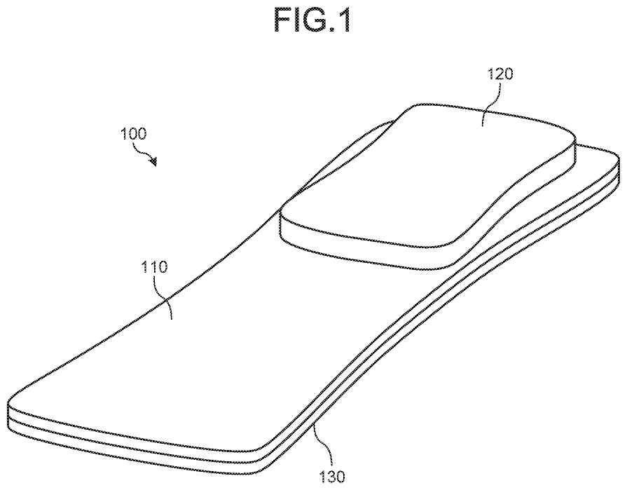

The following describes an exemplary structure of a wearable device 100 according to the embodiment with reference to FIG. 1. FIG. 1 is a diagram illustrating an exemplary structure of the wearable device 100 according to the embodiment. FIG. 1 illustrates a perspective view of the wearable device 100.

The wearable device 100 is a device (sensor device) configured to acquire living body information of a subject. For example, the wearable device 100 is formed in a substantially rectangular (strip) shape and bonded to skin of the subject when used. The subject is a person from which living body information is acquired and corresponds to a person (wearer) who wears the wearable device 100. A “substantially rectangular shape” includes not only a rectangular shape with four corners (apexes) and parallel facing sides but also, for example, a shape with four rounded corners and slightly curved sides as illustrated in FIG. 1.

For example, the wearable device 100 detects an electrocardiogram as living body information. The following description is made on the wearable device 100 configured to acquire an electrocardiogram, but the embodiment is not limited thereto. For example, the wearable device 100 may be a sensor device configured to acquire living body information other than an electrocardiogram, such as body temperature or blood pressure. The wearable device 100 may acquire not only a single kind of living body information but also a plurality of kinds of living body information.

As illustrated in FIG. 1, the wearable device 100 includes a flexible substrate 110, an electronic component unit 120, and an adhesive unit 130. The flexible substrate 110, the electronic component unit 120, and the adhesive unit 130 will be described in detail later.

Contents described with reference to FIG. 1 are merely exemplary and not limited to illustrated contents. For example, FIG. 1 exemplifies the case in which the wearable device 100 has a substantially rectangular shape, but the embodiment is not limited thereto. The wearable device 100 may be formed in an optional shape such as a square, circular, or elliptical shape. The wearable device 100 preferably has a substantially rectangular or elliptical shape among these shapes, and in particular, preferably has a substantially rectangular or elliptical shape having an aspect ratio (ratio of the major radius relative to the minor radius) equal to or larger than 1.1. The wearable device 100 of such a shape can have a large inter-electrode distance per area and thus can acquire continuous living body information at higher sensitivity. Moreover, each end part shape of the wearable device 100 is preferably not angulated. With such a shape, the wearable device 100 is less likely to be separated from skin of the subject and provide discomfort.

In the present embodiment, a direction departing from skin of the subject when the wearable device 100 is bonded to the skin is referred to as “up” in some cases. The direction “up” is an example of a first direction. In addition, a direction opposite to “up” is referred to as “down” in some cases. The direction “down” is an example of a second direction.

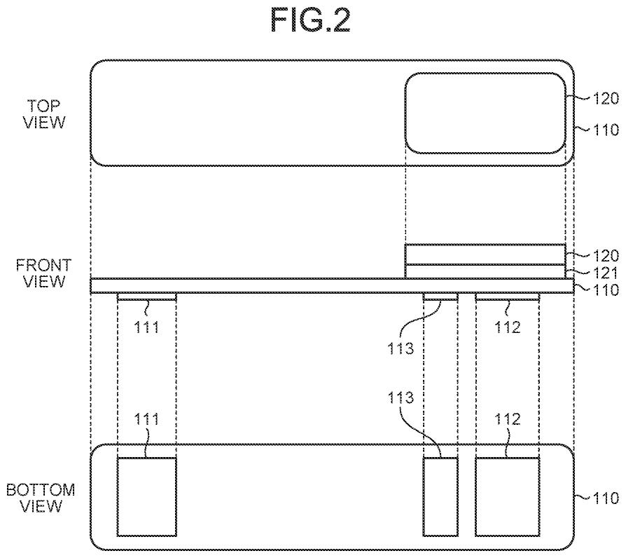

The following describes an exemplary structure of the flexible substrate 110 and the electronic component unit 120 according to the embodiment with reference to FIG. 2. FIG. 2 is a diagram illustrating an exemplary structure of the flexible substrate 110 and the electronic component unit 120 according to the embodiment. The upper part of FIG. 2 exemplifies a top view of the flexible substrate 110 and the electronic component unit 120. The middle part of FIG. 2 exemplifies a front view of the flexible substrate 110 and the electronic component unit 120. The lower part of FIG. 2 exemplifies a bottom view of the flexible substrate 110 and the electronic component unit 120.

The flexible substrate 110 is a conductor-wired substrate having flexibility with which the flexible substrate 110 can flexibly follow motion of a person wearing the wearable device 100. The flexible substrate 110 is, for example, a flexible printed circuit (FPC) formed in a substantially rectangular shape. The flexible substrate 110 may be formed of an optional material such as polyimide resin, polyamide resin, polyester resin, epoxy resin, or acrylic resin. The flexible substrate 110 preferably has a thickness equal to or larger than 0.10 mm and smaller than 0.80 mm, more preferably has a thickness equal to or larger than 0.15 mm and smaller than 0.60 mm. A layer including the flexible substrate 110 is referred to as a “flexible layer”.

For example, a conductor that electrically connects electronic components mounted on the wearable device 100 is provided on a first surface (upper surface) of the flexible substrate 110. Three electrodes 111, 112, and 113 are provided on a second surface (bottom surface) opposite the first surface of the flexible substrate 110. Living body information can be more reliably acquired when the electrodes 111 and 112 are used as a positive electrode and a negative electrode and the electrode 113 positioned between the positive electrode and the negative electrode is used as a reference electrode.

The electrodes 111, 112, and 113 are electrodes for detecting an electrocardiogram (ECG) of the subject. Each of the electrodes 111, 112, and 113 is electrically connected to, through a through-hole, a conductor wired on the upper surface of the flexible substrate 110. A layer including the three electrodes 111, 112, and 113 is referred to as an “electrode layer”. The electrode layer is an exemplary “detection unit” (or detector) configured to detect living body information.

FIG. 2 illustrates the case in which the number of electrodes for electrocardiogram detection is three, but the embodiment is not limited thereto. The number of electrodes for electrocardiogram detection may be set as necessary.

The electronic component unit 120 is electrically connected to the conductor wired on the upper surface of the flexible substrate 110 and includes various electronic components that control processing at the wearable device 100. For example, the electronic component unit 120 controls power supply to each component in the wearable device 100, amplification and collection of an electrocardiogram detected by the electrodes 111, 112, and 113, and outputting of the collected electrocardiogram. A layer including the electronic component unit 120 is referred to as an “electronic component layer”.

The electronic component unit 120 is stacked on a hard substrate 121. The hard substrate 121 is stacked on the flexible substrate 110 and supports the electronic component unit 120. For example, a hard substrate such as a paper phenol substrate, a paper epoxy substrate, a glass composite substrate, a glass epoxy substrate, or a glass polyimide substrate is optionally applicable as the hard substrate 121. Among such hard substrates, the paper epoxy substrate, the glass epoxy substrate, and the glass polyimide substrate are preferable, and the glass epoxy substrate is more preferable, in particular. The hard substrate 121 preferably has a thickness equal to or larger than 0.30 mm and smaller than 3.0 mm, more preferably has a thickness equal to or larger than 0.50 mm and smaller than 2.0 mm. The electronic component unit 120 is more securely supported by such a hard substrate, and thus living body information can be more reliably acquired.

When stacked on the hard substrate 121, the electronic component unit 120 has improved durability against deflection (bending) as compared to a configuration in which the hard substrate 121 is directly stacked on the flexible substrate 110 and a configuration in which the hard substrate 121 is installed inside the flexible substrate 110. As a result, the wearable device 100 reduces the risk of damage that soldering of the electronic component unit 120 comes off by deflection and wire breaking occurs, and thus can reliably acquire continuous living body information.

The hard substrate 121 is disposed at an end part of the flexible substrate 110. Accordingly, the number of wires on the flexible substrate 110 is reduced to several wires to the electronic component unit 120 (hard substrate 121), and thus wiring is simplified. As a result, the wearable device 100 reduces the risk of wire breaking and thus can reliably acquire continuous living body information.

The hard substrate 121 is disposed at a position corresponding to the electrode 112 of the flexible substrate 110, and more preferably disposed at a position corresponding to the electrodes 112 and 113. Accordingly, the electrode 112 and the like are robust against deflection. As a result, the wearable device 100 reduces the risk of damage on the electrode 112 and the like by deflection and thus can reliably acquire continuous living body information.

The area of the hard substrate 121 is preferably 5 to 50% of the area of the flexible substrate 110, more preferably 20 to 40% of the area of the flexible substrate 110. When the area of the hard substrate 121 is in such a range, damage on the wearable device 100 is unlikely to occur and the electronic component unit 120 is more securely be supported, and thus living body information can be more reliably acquired. The area of the hard substrate 121 is the area of a surface of the hard substrate 121 facing the flexible substrate 110. The area of the flexible substrate 110 is the area of a surface of the flexible substrate 110 facing the hard substrate 121.

Contents described with reference to FIG. 2 are merely exemplary and not limited to illustrated contents. For example, FIG. 2 illustrates the case in which the electronic component unit 120 is stacked on the hard substrate 121, but the embodiment is not limited thereto. The electronic component unit 120 may be stacked on an optional member that is harder (less likely to be deflected) than the flexible substrate 110. In other words, the electronic component unit 120 is stacked on an “isolation layer” for isolation from deflection of the flexible substrate 110. For example, a flexible substrate made of a material harder than the flexible substrate 110 or a flexible substrate formed thicker than the flexible substrate 110 is optionally applicable as the isolation layer. The isolation layer is referred to as a “support layer” supporting the electronic component unit 120.

The material of each component described with reference to FIG. 2 is not limited to the above-described material. For example, other materials may optionally be used as the material of each of the flexible substrate 110, the electrodes 111, 112, and 113, the electronic component unit 120, and the hard substrate 121 to the extent not impairing functions of each above-described component.

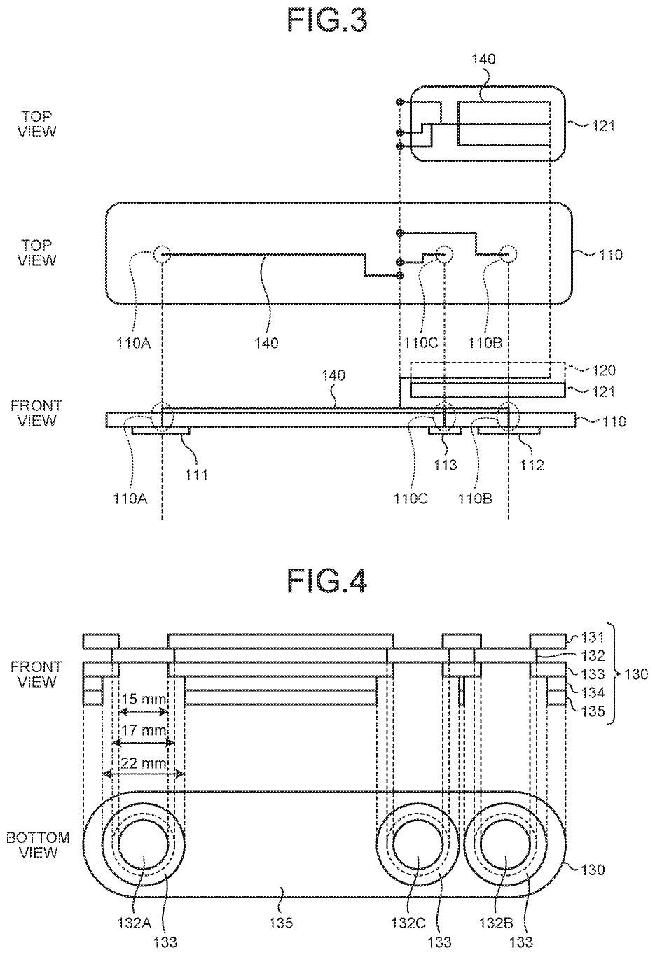

The following describes exemplary wiring on the flexible substrate 110 and the hard substrate 121 according to the embodiment with reference to FIG. 3. FIG. 3 is a diagram illustrating exemplary wiring on the flexible substrate 110 and the hard substrate 121 according to the embodiment. The upper part of FIG. 3 exemplifies wiring in a top view of the hard substrate 121. The middle part of FIG. 3 exemplifies wiring in a top view of the flexible substrate 110. The lower part of FIG. 3 exemplifies wiring in a front view of the flexible substrate 110 and the hard substrate 121.

As illustrated in FIG. 3, a plurality of lead lines 140 are disposed on the upper surface of the hard substrate 121. The lead lines 140 electrically connect various electronic components included in the electronic component unit 120. Wiring bifurcated into three, which is illustrated in the upper part of FIG. 3, is merely exemplary, and the embodiment is not limited thereto. The lead lines 140 on the hard substrate 121 are insulated as appropriate and connected to the lead lines 140 on the flexible substrate 110.

The lead lines 140 for connecting the three electrodes 111, 112, and 113 to the electronic component unit 120 are disposed on the upper surface of the flexible substrate 110. Through-holes are formed at respective positions 110A, 110B, and 110C on the flexible substrate 110. The lead lines 140 is connected to the three electrodes 111, 112, and 113 through the through-holes formed at the respective positions. The lead lines 140 extending from the electrodes 111, 112, and 113 are insulated and individually connected to the lead lines 140 on the hard substrate 121.

Contents described with reference to FIG. 3 are merely exemplary and not limited to illustrated contents. For example, the lower part of FIG. 3 (front view) illustrates that the lead lines 140 disposed on the upper surface of each substrate are separated from the substrate, but this configuration is intended to clearly illustrate the lead lines 140 and the members are not separated from each other in reality.

FIG. 3 illustrates the case in which the lead lines 140 on the hard substrate 121 are bundled at “one place”, but the lead lines 140 may be bundled at several places and connected to the flexible substrate 110. However, the number of bundling places is preferably small for wiring simplification.

The following describes an exemplary structure of the adhesive unit 130 according to the embodiment with reference to FIG. 4. FIG. 4 is a diagram illustrating an exemplary structure of the adhesive unit 130 according to the embodiment. The upper part of FIG. 4 exemplifies a front view of the adhesive unit 130. The lower part of FIG. 4 exemplifies a bottom view of the adhesive unit 130.

The adhesive unit 130 is disposed on the bottom surface of the flexible substrate 110 and has adhesive force for bonding the flexible substrate 110 and the electronic component unit 120 to skin of the subject. The adhesive unit 130 includes five layers of a waterproof layer 131, a conductive gel layer 132, a waterproof layer 133, a water-absorbing layer 134, and a skin bonding layer 135.

The waterproof layer 131 is a waterproof and insulating layer disposed on the bottom surface of the flexible substrate 110 (electrode layer). For example, the waterproof layer 131 contains a material having an excellent waterproof property, such as polyester resin, polyurethane resin, polyethylene resin, polypropylene resin, or nylon resin. The waterproof layer 131 has holes through which the electrodes 111, 112, and 113 contact conductive gels 132A, 132B, and 132C to be described later. In the example illustrated in FIG. 4, the waterproof layer 131 has circular holes having a diameter of 15 mm.

The conductive gel layer 132 is disposed on the bottom surface of the waterproof layer 131, can contact skin of the subject, and are electrically connected to the electrodes 111, 112, and 113. For example, the conductive gel layer 132 includes the three conductive gels 132A, 132B, and 132C. The conductive gel layer 132 is an exemplary “detection unit” (or detector) configured to detect living body information.

The conductive gels 132A, 132B, and 132C are conductive hydrogels (aqueous gels). In the example illustrated in FIG. 4, the conductive gels 132A, 132B, and 132C each have a circular shape having a diameter of 17 mm. The conductive gel 132A is disposed on the bottom surface of the electrode 111 and electrically connected to the electrode 111. The conductive gel 132B is disposed on the bottom surface of the electrode 112 and electrically connected to the electrode 112. The conductive gel 132C is disposed on the bottom surface of the electrode 113 and electrically connected to the electrode 113.

No member or an optional member may be disposed in a region on the conductive gel layer 132 in which the three conductive gels 132A, 132B, and 132C do not exist. When no member is disposed, the waterproof layer 131 and the waterproof layer 133 are directly bonded to each other. When an optional member is disposed, an insulating and waterproof member is preferably disposed.

The waterproof layer 133 is a waterproof and insulating layer disposed on the bottom surface of the conductive gel layer 132. For example, the waterproof layer 133 contains a material having an excellent waterproof property, such as polyester resin, polyurethane resin, or nylon resin. The waterproof layer 133 has holes through which the conductive gels 132A, 132B, and 132C contact skin of the subject. In the example illustrated in FIG. 4, the waterproof layer 133 has circular holes having a diameter of 15 mm. The bottom surface of the conductive gel layer 132 corresponds to a surface of the conductive gel layer 132 that is opposite a surface adjacent to the electrode layer.

The water-absorbing layer 134 is a water-absorbing layer disposed on the bottom surface of the waterproof layer 133. For example, the water-absorbing layer 134 contains non-woven fabric. The water-absorbing layer 134 is not limited to the non-woven fabric but may be made of, for example, a water-absorbing fiber. For example, polyester resin, polyurethane resin, polypropylene resin, or nylon resin is preferably used as the material of such a fiber, and in particular, polyurethane resin or polypropylene resin is preferably used as the material. The fiber of such a material is excellent in ventilation, expansion, and contraction, and thus an effect of the water-absorbing layer 134 to be described later is likely to be exerted. The bottom surface of the waterproof layer 133 corresponds to a surface of the waterproof layer 133 that is opposite a surface adjacent to the conductive gel layer 132.

The water-absorbing layer 134 has holes through which the conductive gels 132A, 132B, and 132C contact skin of the subject. In the example illustrated in FIG. 4, the water-absorbing layer 134 has circular holes having a diameter of 22 mm.

The water-absorbing layer 134 has an effect of absorbing sweat (water) generated on skin of the subject and exhaling the sweat to the outside of the wearable device 100. Accordingly, the water-absorbing layer 134 prevents the hydrogel (the conductive gel layer 132) from absorbing water and expanding and can reduce discomfort due to expansion. Moreover, with hydrogel expansion prevention, the water-absorbing layer 134 reduces the probability that the hydrogel separates from an adjacent layer. Accordingly, the probability that the wearable device 100 is separated from skin of the wearer due to sweat can be reduced, and furthermore, the wearable device 100 can be continuously stuck on skin of the wearer for about two weeks at longest.

With the water-absorbing layer 134, the wearable device 100 can reduce influence on a human body (skin), such as cell toxicity, sensitization (allergy reaction), and irritation. In particular, the influence on a human body can be further reduced when non-woven fabric is employed as the water-absorbing layer 134.

Each hole of the water-absorbing layer 134 is larger than each hole of the waterproof layer 133. Accordingly, end parts (rims) of holes of the water-absorbing layer 134 are separated from the corresponding conductive gels 132A, 132B, and 132C, and thus it is possible to reduce the probability that water absorbed by the water-absorbing layer 134 is absorbed by the conductive gels 132A, 132B, and 132C through the end parts of the holes.

The skin bonding layer 135 is disposed on the bottom surface of the water-absorbing layer 134 and can be bonded to skin of the subject. For example, the skin bonding layer 135 contains an acrylic bonding agent or hydrocolloid. The skin bonding layer 135 has holes through which the conductive gels 132A, 132B, and 132C contact skin of the subject. In the example illustrated in FIG. 4, the waterproof layer 133 has circular holes having a diameter of 22 mm.

Contents described with reference to FIG. 4 are merely exemplary and not limited to illustrated contents. For example, the shape and size of the conductive gel layer 132 are not limited to illustrated contents but may be optionally changed. The shapes and sizes of holes (or grooves) of the waterproof layer 131, the waterproof layer 133, the water-absorbing layer 134, and the skin bonding layer 135 are changed in accordance with change of the shape and size of the conductive gel layer 132. The holes (or grooves) of the waterproof layer 131, the waterproof layer 133, the water-absorbing layer 134, and the skin bonding layer 135 are exemplary opening parts.

Although not illustrated in FIG. 4, the adhesive unit 130 may include, in addition to the above-described five layers, bonding layers for bonding the layers. Each bonding layer may be an optional bonding agent of liquid, a sheet, or the like. However, the bonding layers may be omitted when each above-described layer has a function to bond to an adjacent layer. For example, a bonding layer between the water-absorbing layer 134 and the skin bonding layer 135 may be omitted when the skin bonding layer 135 has a function to bond to the water-absorbing layer 134. A bonding layer between the waterproof layer 133 and the water-absorbing layer 134 may be omitted when the waterproof layer 133 has a function to bond to the water-absorbing layer 134.

The material of each component described with reference to FIG. 4 is not limited to the above-described material. For example, other materials may optionally be used as the material of each of the waterproof layer 131, the conductive gel layer 132, the waterproof layer 133, the water-absorbing layer 134, and the skin bonding layer 135 to the extent not impairing functions of each above-described component.

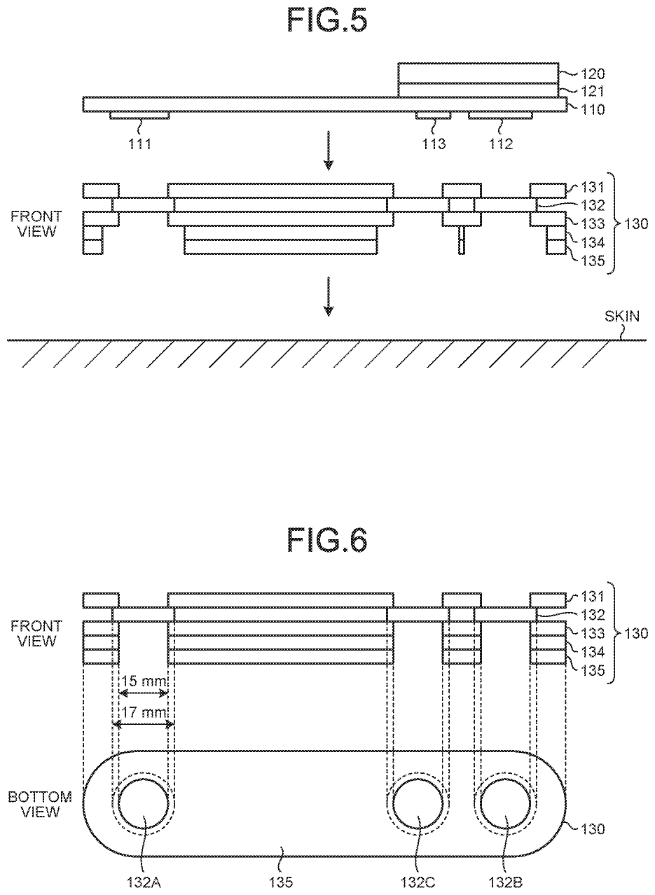

The following describes a bonding method for the wearable device 100 according to the embodiment with reference to FIG. 5. FIG. 5 is a diagram for description of the bonding method for the wearable device 100 according to the embodiment.

For example, the wearable device 100 is distributed in a state in which the flexible substrate 110, the electronic component unit 120, and the adhesive unit 130 are integrated. Specifically, as illustrated in FIG. 5, the flexible substrate 110 and the electronic component unit 120 are bonded to the upper surface of the adhesive unit 130 in advance. The upper surface of the adhesive unit 130 corresponds to the upper surface of the waterproof layer 131 that is opposite a surface adjacent to the conductive gel layer 132.

Although not illustrated in FIG. 5, the bottom surface of the wearable device 100 (that is, the bottom surface of the skin bonding layer 135) is covered by release paper. A user of the wearable device 100 peels the release paper to expose the bottom surface of the skin bonding layer 135. Then, the user bonds the bottom surface of the skin bonding layer 135 to an optional position on skin of a subject. The bottom surface of the adhesive unit 130 corresponds to the bottom surface of the skin bonding layer 135 that is opposite a surface adjacent to the water-absorbing layer 134. The user of the wearable device 100 corresponds to, for example, a medical professional such as a doctor, the subject, or a person who supports the subject at a medical aspect or a living aspect.

Specifically, the wearable device 100 has a layer configuration including, sequentially from a side close to a human body (skin), the skin bonding layer 135, the water-absorbing layer 134, the waterproof layer 133, the conductive gel layer 132, the waterproof layer 131, the electrode layer (electrodes 111, 112, and 113), the flexible layer (flexible substrate 110), the isolation layer (hard substrate 121), and the electronic component layer (electronic component unit 120). The electrode part (electrodes 111, 112, and 113) of the wearable device 100 bonded to an optional position on the skin detects living body information from the human body through the conductive gel part (132).

Contents described with reference to FIG. 5 are merely exemplary and not limited to illustrated contents. For example, although FIG. 5 illustrates, as a representative example, distribution in a state in which the flexible substrate 110, the electronic component unit 120, and the adhesive unit 130 are integrated, the embodiment is not limited thereto. For example, each component included in the wearable device 100 may be manufactured and distributed by an individual business operator.

For convenience of illustration, each layer has the same thickness in FIG. 5, but the thickness of each layer may be optionally set. Since the thicknesses of the skin bonding layer 135, the water-absorbing layer 134, and the waterproof layer 133 are extremely smaller than the sizes (e.g., diameters) of holes of the skin bonding layer 135, the water-absorbing layer 134, and the waterproof layer 133, the conductive gel layer 132 easily contacts skin of the subject when the wearable device 100 is bonded to skin of the subject.

The layer including the three electrodes 111, 112, and 113 is referred to as an “electrode layer” in the above-described embodiment but does not necessarily need to be referred to as a “layer” because the electrodes 111, 112, and 113 are smaller than the flexible layer and the isolation layer and provided at points. For example, the three electrodes 111, 112, and 113 may be referred to as an “electrode part”.

The layer including the three conductive gels 132A, 132B, and 132C is referred to as a “conductive gel layer” in the above-described embodiment but does not necessarily need to be referred to as a “layer” because the conductive gels 132A, 132B, and 132C are smaller than the flexible layer and the isolation layer and provided at points. For example, the three conductive gels 132A, 132B, and 132C may be referred to as a “conductive gel part”.

As described above, the wearable device 100 includes the detection unit (e.g., a detector comprising electrode layer and conductive gel layer 132), the flexible layer (flexible substrate 110), the isolation layer (hard substrate 121), and the electronic component layer (the electronic component unit 120). The detection unit detects living body information. The flexible layer includes a conductor electrically connected to the detection unit. The isolation layer is stacked on the flexible layer and is harder than the flexible layer. The electronic component layer is stacked on the isolation layer and includes an electronic component electrically connected to the conductor. With this configuration, the wearable device 100 can reliably acquire continuous living body information. For example, the wearable device 100 includes the isolation layer for isolating the electronic component layer from deflection of the flexible layer, reduces the risk of damage on the electronic component layer due to deflection, and thus can reliably acquire continuous living body information.

In other words, the wearable device 100 includes the detection unit, a first layer, a second layer, and a third layer. The detection unit detects living body information. The first layer is flexible and disposed in the first direction relative to the detection unit and includes a conductor electrically connected to the detection unit. The second layer is disposed in the first direction relative to the first layer and is harder than the first layer. The third layer is disposed in the first direction relative to the second layer and includes an electronic component electrically connected to the conductor. The wearable device 100 also includes a fourth layer and a fifth layer. The fourth layer has a waterproof property and is disposed in the second direction opposite the first direction relative to the detection unit. The fifth layer has a water-absorbing property and disposed in the second direction relative to the fourth layer. The above notation of “first” to “fifth” is used to distinguish the layers and does not mean an order or the like.

The wearable device 100 also includes the conductive gel layer 132, the electrode layer (electrodes 111, 112, and 113), the electronic component layer (electronic component unit 120), the flexible layer (flexible substrate 110), the waterproof layer 133, and the water-absorbing layer 134. The conductive gel layer 132 can contact skin of the subject. The electrode layer includes an electrode electrically connected to the conductive gel layer 132. The electronic component layer includes an electronic component. The electrode layer is disposed on the first surface of the flexible layer, the electronic component layer is disposed on the second surface opposite the first surface, and the flexible layer includes a conductor that electrically connects the electrode layer and the electronic component layer. The waterproof layer 133 is disposed on the surface of the conductive gel layer 132 that is opposite a surface adjacent to the electrode layer. The water-absorbing layer 134 is disposed on the surface of the waterproof layer 133 that is opposite a surface adjacent to the conductive gel layer 132. With this configuration, the wearable device 100 can reliably acquire continuous living body information. For example, the wearable device 100, which includes the water-absorbing layer 134 that absorbs sweat and exhales the sweat to the outside of the wearable device 100, reduces the probability that the hydrogel (conductive gel layer 132) absorbs water, expands, and separates from an adjacent layer. As a result, the wearable device 100 can reliably acquire continuous living body information.

In other words, the wearable device 100 includes the first layer, the second layer, the conductive gel part, the electrode part, the third layer, and the fourth layer. The first layer has a water-absorbing property. The second layer has a waterproof property and is disposed in the first direction relative to the first layer. The conductive gel part is disposed in the first direction relative to the second layer and can contact skin of the subject. The electrode part is disposed in the first direction relative to the conductive gel part and electrically connected to the conductive gel part. The third layer is flexible and disposed in the first direction relative to the electrode part and includes a conductor electrically connected to the electrode part. The fourth layer is disposed in the first direction relative to the third layer and includes an electronic component electrically connected through the conductor. The above notation “first” to “fourth” is used to distinguish the layers and does not mean an order or the like.

Modification

In the above-described embodiment, each hole of the water-absorbing layer 134 is larger than each hole of the waterproof layer 133, but the embodiment is not limited thereto. For example, the size of each hole of the water-absorbing layer 134 may be the same as that of each hole of the waterproof layer 133.

The following describes an exemplary structure of the adhesive unit 130 according to a modification with reference to FIG. 6. FIG. 6 is a diagram illustrating an exemplary structure of the adhesive unit 130 according to the modification. The upper part of FIG. 6 exemplifies a front view of the adhesive unit 130. The lower part of FIG. 6 exemplifies a bottom view of the adhesive unit 130.

As illustrated in FIG. 6, each hole of the water-absorbing layer 134 has a diameter of 15 mm, which is the same size as that of each hole of the waterproof layer 133. In this case, the end parts (rims) of the holes of the water-absorbing layer 134 are near the conductive gels 132A, 132B, and 132C, and thus water absorbed by the water-absorbing layer 134 is potentially absorbed by the conductive gels 132A, 132B, and 132C through the end parts of the holes.

To avoid this, the waterproof layer 133 has a thickness equal to or larger than 30 μm and smaller than 150 μm, more preferably has a thickness equal to or larger than 75 μm and smaller than 120 μm. In this manner, the end parts (rims) of the holes of the water-absorbing layer 134 are separated from the conductive gels 132A, 132B, and 132C when the waterproof layer 133 has a certain thickness or larger, and thus it is possible to reduce the probability that water absorbed by the water-absorbing layer 134 is absorbed by the conductive gels 132A, 132B, and 132C through the end parts of the holes. Moreover, the wearable device 100 more easily follows motion of the wearer when the waterproof layer 133 has a certain thickness or smaller, and thus it is possible to more reliably acquire living body information.

In the example illustrated in FIG. 6, each hole of the skin bonding layer 135 has a diameter of 15 mm, which is the same size as those of each hole of the waterproof layer 133 and each hole of the water-absorbing layer 134. Accordingly, the holes can be formed after the waterproof layer 133, the water-absorbing layer 134, and the skin bonding layer 135 are superimposed, which leads to a simplified manufacturing process.

### OTHER EMBODIMENTS

The present invention may be performed in various kinds of different forms other than the above-described embodiment.

Detection Method

The present embodiment may be provided as a detection method including detecting an anomaly (heart disease such as atrial fibrillation) of the subject based on living body information acquired by using the wearable device 100. For example, the detection method detects an anomaly of the subject based on the living body information for a duration of time, e.g., 72 hours or longer. Detection of an anomaly of the subject can be performed at higher accuracy as such the detection duration increases, and may be performed, for example, based on a detection duration of 120 hours or longer. In another embodiment, the detection duration may be 168 hours or longer. Various methods may optionally be used as the method of detecting an anomaly based on living body information.

The above-described embodiment may be optionally combined with the above-described modifications, or the above-described modifications may be optionally combined with each other.

### EXAMPLES

The present invention will be described below in more detail based on examples but is not limited to the following description.

Wearable devices corresponding to first to seventh examples and first and second comparative examples, respectively, listed in Table 1 were produced based on the configuration described above in the embodiment. Table 1 lists device configurations and evaluation results of the wearable devices according to the examples and the comparative examples.

TABLE 1

| | | | | | | | | | | First | Second |
|---|---|---|---|---|---|---|---|---|---|---|---|
| | | | | | | | | | | compar- | compar- |
| | | | First | Second | Third | Fourth | Fifth | Sixth | Seventh | ative | ative |
| | | | example | example | example | example | example | example | example | example | example |
| Device | Layer | Skin bonding layer | ∘ | ∘ | ∘ | ∘ | ∘ | ∘ | ∘ | ∘ | ∘ |
| configuration | configuration | Water-absorbing | ∘ | ∘ | ∘ | ∘ | ∘ | ∘ | — | — | — |
| | | layer | | | | | | | | | |
| | | Waterproof layer | ∘ | ∘ | ∘ | ∘ | ∘ | ∘ | ∘ | ∘ | — |
| | | Conductive gel | ∘ | ∘ | ∘ | ∘ | ∘ | ∘ | ∘ | ∘ | ∘ |
| | | layer | | | | | | | | | |
| | | Waterproof layer | ∘ | ∘ | ∘ | ∘ | ∘ | ∘ | ∘ | ∘ | ∘ |
| | | Electrode layer | ∘ | ∘ | ∘ | ∘ | ∘ | ∘ | ∘ | ∘ | ∘ |
| | | Flexible layer | ∘ | ∘ | ∘ | ∘ | ∘ | ∘ | ∘ | ∘ | ∘ |
| | | Isolation layer | ∘ | ∘ | ∘ | ∘ | ∘ | ∘ | ∘ | — | — |
| | | Electronic | ∘ | ∘ | ∘ | ∘ | ∘ | ∘ | ∘ | ∘ | ∘ |
| | | component layer | | | | | | | | | |

| | | | | | | | | | | |
|---|---|---|---|---|---|---|---|---|---|---|
| | Isolation layer area | 35% | 15% | 45% | 35% | 35% | 35% | 35% | — | — |
| | Isolation layer material | Glass | Glass | Glass | Paper | Glass | Glass | Glass | — | — |
| | | epoxy | epoxy | epoxy | phenol | polyimide | epoxy | epoxy | | |
| | Isolation layer position | End part | End part | End part | End part | End part | Center | End | — | — |
| Evaluation | Bending test 1 | A | B | A | A | A | A | A | B | B |
| results | Bending test 2 | A | B | A | B | A | B | A | C | C |
| | Data accuracy | A | A | B | A | B | B | A | A | B |
| | Waterproof property | A | A | A | A | A | A | A | A | B |
| | Discomfort | A | A | B | A | B | B | B | B | C |

In the example of Table 1, the wearable devices according to the examples and the comparative examples were produced by changing device configurations. The changed device configurations are four items of a layer configuration, an isolation layer area, an isolation layer material, and an isolation layer position.

The layer configuration is the layer configuration described above in the embodiment. Specifically, the layer configuration listed in Table 1 indicates whether the skin bonding layer 135, the water-absorbing layer 134, the waterproof layer 133, the conductive gel layer 132, the waterproof layer 131, the electrode layer (electrodes 111, 112, and 113), the flexible layer (flexible substrate 110), the isolation layer (hard substrate 121), and the electronic component layer (electronic component unit 120) are provided sequentially from a side close to a human body (skin). In Table 1, a symbol “o” indicates that the corresponding layer is provided, and a symbol “-” indicates that the corresponding layer is not provided.

As for a detailed configuration (material) of each layer, a layer containing an acrylic bonding agent and hydrocolloid was used as the skin bonding layer 135, non-woven fabric made of polyurethane resin was used as the water-absorbing layer 134, a layer made of polyester resin was used as the waterproof layer 133 and the waterproof layer 131, and a substrate made of polyimide resin and having a thickness of 0.18 mm was used as the flexible substrate 110. In the electrode layer, as illustrated in FIGS. 2 and 5, the electrodes 111 and 112 were disposed at end parts of the corresponding wearable device in the longitudinal direction, and the electrode 113 was disposed at a position close to the electrode 112 between the two electrodes. The electrodes 111 and 112 were used a positive electrode and the negative electrode, and the electrode 113 was used as a reference electrode. The isolation layer is made of a material to be described later with reference to “isolation layer material” in Table 1 and has a thickness of 0.8 mm.

The isolation layer area indicates the ratio [%] of the area of the isolation layer relative to the area of the flexible layer. The symbol “-” indicates that no corresponding data is available (no isolation layer is provided).

The isolation layer material indicates the material of the isolation layer. In the example of Table 1, any of glass epoxy, paper phenol, and glass polyimide was selected as the isolation layer material. The symbol “-” indicates that no corresponding data is available (no isolation layer is provided).

The isolation layer position indicates the position of the isolation layer on the flexible layer. In the example of Table 1, an end part or the center was selected as the isolation layer position. The end part is one of the end parts of the corresponding wearable device in the longitudinal direction and is a position corresponding to the electrode 111 or the electrode 112. The center is a position that is between the electrode 111 and the electrode 112 and does not correspond to the electrode 113. The symbol “-” indicates that no corresponding data is available (no isolation layer is provided).

As indicated in Table 1, the wearable device of the first example includes the nine layers of the skin bonding layer 135, the water-absorbing layer 134, the waterproof layer 133, the conductive gel layer 132, the waterproof layer 131, the electrode layer, the flexible layer, the isolation layer, and the electronic component layer. As for the wearable device of the first example, the isolation layer area is “35%”, the isolation layer material is “glass epoxy”, and the isolation layer position is “end part”.

As for the wearable device of the second example, the isolation layer area is “15%”. The layer configuration, the isolation layer material, and the isolation layer position of the wearable device of the second example are the same as the layer configuration, the isolation layer material, and the isolation layer position of the wearable device of the first example.

As for the wearable device of the third example, the isolation layer area is “45%”. The layer configuration, the isolation layer material, and the isolation layer position of the wearable device of the third example are the same as the layer configuration, the isolation layer material, and the isolation layer position of the wearable device of the first example.

As for the wearable device of the fourth example, the isolation layer material is “paper phenol”. The layer configuration, the isolation layer area, and the isolation layer position of the wearable device of the fourth example are the same as the layer configuration, the isolation layer area, and the isolation layer position of the wearable device of the first example.

As for the wearable device of the fifth example, the isolation layer material is “glass polyimide”. The layer configuration, the isolation layer area, and the isolation layer position of the wearable device of the fifth example are the same as the layer configuration, the isolation layer area, and the isolation layer position of the wearable device of the first example.

As for the wearable device of the sixth example, the isolation layer position is “center”. The layer configuration, the isolation layer area, and the isolation layer material of the wearable device of the sixth example are the same as the layer configuration, the isolation layer area, and the isolation layer material of the wearable device of the first example.

The wearable device of the seventh example includes the eight layers of the skin bonding layer 135, the waterproof layer 133, the conductive gel layer 132, the waterproof layer 131, the electrode layer, the flexible layer, the isolation layer, and the electronic component layer without the layer configuration in the water-absorbing layer 134. The isolation layer area, the isolation layer material, and the isolation layer position of the wearable device of the seventh example are the same as the isolation layer area, the isolation layer material, and the isolation layer position of the wearable device of the first example.

The wearable device of the first comparative example includes the seven layers of the skin bonding layer 135, the waterproof layer 133, the conductive gel layer 132, the waterproof layer 131, the electrode layer, the flexible layer, and the electronic component layer without the water-absorbing layer 134 and the isolation layer in the layer configuration. Since the wearable device of the first comparative example includes no isolation layer, no data is available for the isolation layer area, the isolation layer material, and the isolation layer position.

The wearable device of the second comparative example includes the six layers of the skin bonding layer 135, the conductive gel layer 132, the waterproof layer 131, the electrode layer, the flexible layer, and the electronic component layer without the water-absorbing layer 134, the waterproof layer 133, and the isolation layer in the layer configuration. Since the wearable device of the second comparative example includes no isolation layer, no data is available for the isolation layer area, the isolation layer material, and the isolation layer position.

The following describes evaluation of the wearable devices according to the examples and the comparative examples. The wearable devices according to the examples and the comparative examples were evaluated for five items of a bending test 1, a bending test 2, data accuracy, waterproof property, and discomfort.

In the bending test 1, each wearable device is bent and stretched 100 times by using a test device for the bending test. The test device can automatically repeat bending and stretching of a test target object in a plate shape. The test device includes, as a base, two plate members having a variable relative angle therebetween and performs bending and stretching of a test target object with respective ends of the test target object being fixed to the two plate members. In the present example, a part of 15 mm at each end of the wearable device was fixed to the base with a tape, and bending and stretching with the bending radius of 17 mm and the bending angle of 180° were repeatedly performed. After the 100 times of bending and stretching, the wearable device was used and the damage status thereof was evaluated. The damage status was evaluated at three levels of “A”, “B”, and “C”. The level “A” indicates that the wearable device can be used as before the test, in other words, no damage was observed. The level “B” indicates that the wearable device operated but a partial loss of acquired data was observed. The level “C” indicates that the wearable device did not operate.

In the bending test 2, each wearable device was bent and stretched 1000 times by using the test device used in the bending test 1. After the 1000 times of bending and stretching, the wearable device was used and the damage status thereof was evaluated. The reference of evaluation of the damage status is the same as that of the bending test 1 and thus description thereof is omitted.

As for the data accuracy, the accuracy of data (electrocardiogram) acquired while each wearable device was used for seven days was evaluated based on the amount of data loss and noise. The data accuracy was evaluated at three levels of “A”, “B”, and “C”. The level “A” indicates that no data loss and noise were observed. The level “B” indicates that loss and noise were observed at part of the data. The level “C” indicates that the wearable device was damaged and data for the seven days was not obtained.

The waterproof property was evaluated based on the expansion rate of the conductive gel layer 132 after each wearable device was left for seven days in an environment at the temperature of 30° and the humidity of 95%. The expansion rate was evaluated at two levels of “A” and “B”. The level “A” indicates that the expansion rate is lower than 20%. The level “B” indicates that the expansion rate is equal to or higher than 20%.

The discomfort was measured by evaluating discomfort on a wearer who wore each wearable device for seven days. The discomfort was evaluated at three levels of “A”, “B”, and “C”. The level “A” indicates that no particular discomfort was felt. The level “B” indicates that itching sensation was partially felt. The level “C” indicates that strong itching sensation was felt. Itching sensation occurred in the discomfort evaluation was observed after elapse of substantially 72 hours since wearing. Strong itching sensation was observed after elapse of substantially 120 hours since wearing.

With the wearable device of the first example, evaluation results of “A” were obtained for the five items of the bending test 1, the bending test 2, the data accuracy, the waterproof property, and the discomfort. Most favorable results were obtained for the wearable device of the first example among the wearable devices of the other examples and the comparative example.

With the wearable device of the second example, evaluation results of “B” were obtained for the bending tests 1 and 2. This suggests the risk of wire breaking can be reduced when the isolation layer area has a certain size or larger.

With the wearable device of the third example, evaluation results of “B” were obtained for the data accuracy and the discomfort. This suggests that, when the isolation layer area is reduced, the shape of the wearable device more easily follows body motion and separation can be prevented.

With the wearable device of the fourth example, evaluation results of “B” were obtained for the bending test 2. This suggests that, when the isolation layer has a certain hardness (paper phenol) or larger, the isolation layer can be prevented from bending and the risk of wire breaking can be reduced.

With the wearable device of the fifth example, evaluation results of “B” were obtained for the data accuracy and the discomfort. This suggests that, when the isolation layer has a certain hardness (glass polyimide) or smaller, the shape of the wearable device more easily follows body motion and separation can be prevented.

With the wearable device of the sixth example, evaluation results of “B” were obtained for the bending test 2, the data accuracy, and the discomfort. In a case in which the isolation layer is positioned at a central part, the flexible layer bends at both ends of the isolation layer, and thus the number of bending points increases to two as compared to a case in which the isolation layer is positioned at an end part (the number of bending points is one). This suggests that the risk of wire breaking can be reduced by decreasing the number of bending points. It is also suggested that the capability of following body motion is improved by decreasing the number of bending points and separation can be prevented.

With the wearable device of the seventh example, an evaluation result of “B” was obtained for the discomfort. This suggests that, when the water-absorbing layer is provided, sweat is more likely to be exhaled to the outside of the wearable device and itching sensation can be reduced.

With the wearable device of the first comparative example, evaluation results of “B” were obtained for the bending test 1 and the discomfort, and an evaluation result of “C” was obtained for the bending test 2. This suggests that, when no isolation layer is provided, all important wires for the device are disposed on the flexible layer and the risk of wire breaking increases. It is also suggested that, when no water-absorbing layer is provided, sweat is less likely to be exhaled to the outside of the wearable device, and it could lead to itching sensation.

With the wearable device of the second comparative example, evaluation results of “B” were obtained for the bending test 1, the data accuracy, and the waterproof property, and evaluation results of “C” was obtained for the bending test 2 and the discomfort. This suggests that, when no isolation layer is provided, all important wires for the device are disposed on the flexible layer and the risk of wire breaking increases. It is also suggested that, when no water-absorbing layer 134 is provided, sweat is less likely to be exhaled to the outside of the wearable device, and it could lead to itching sensation. Furthermore, it is suggested that, when neither water-absorbing layer 134 nor waterproof layer 133 is provided, the hydrogel (conductive gel layer 132) absorbs a large amount of sweat generated at the skin bonding surface of the wearable device 100 and largely expands, and the risk of separation increases.

According to the present invention, it is possible to provide a wearable device and a detection method that are capable of reliably acquiring continuous living body information.

Although the invention has been described with respect to specific embodiments for a complete and clear disclosure, the description is not to be thus limited but is to be construed as embodying all modifications and alternative constructions that may occur to one skilled in the art that fairly fall within the basic teaching herein set forth. The embodiments may be combined to form additional embodiments.

What is claimed is:

1. A wearable device, comprising:
a detector configured to detect living body information of a subject;
a first layer that is flexible and disposed in a first direction relative to the detector and includes a conductor electrically connected to the detector;
a second layer that is disposed in the first direction relative to the first layer and is harder than the first layer; and
a third layer that is disposed in the first direction relative to the second layer and includes an electronic component electrically connected to the conductor.

2. The wearable device according to claim 1, wherein the detector includes
an electrode part that includes an electrode electrically connected to the conductor, and
a conductive gel part that is capable of contacting skin of the subject and is electrically connected to the electrode, and
the detector is configured to detect an electrocardiogram as the living body information.

3. The wearable device according to claim 2, further comprising:
a fourth layer that has a waterproof property and is disposed in a second direction opposite the first direction relative to the detector; and
a fifth layer that has a water-absorbing property and is disposed in the second direction relative to the fourth layer.

4. The wearable device according to claim 3, wherein the fourth layer and the fifth layer each have an opening part through which the conductive gel part contacts skin of the subject.

5. The wearable device according to claim 4, wherein the opening part of the fifth layer is larger than the opening part of the fourth layer.

6. The wearable device according to claim 3, wherein the fifth layer includes non-woven fabric.

7. The wearable device according to claim 3, wherein the fourth layer has a thickness equal to or larger than 30 μm and smaller than 150 μm.

8. The wearable device according to claim 7, wherein the fourth layer has a thickness equal to or larger than 75 μm and smaller than 120 μm.

9. The wearable device according to claim 1, wherein an area of the second layer is 5 to 50% of an area of the first layer.

10. The wearable device according to claim 9, wherein the area of the second layer is 15 to 451 of the area of the first layer.

11. The wearable device according to claim 1, wherein the second layer is disposed at an end part of the first layer.

12. The wearable device according to claim 2, wherein the second layer is disposed at a position corresponding to the electrode of the first layer.

13. The wearable device according to claim 1, wherein the first direction corresponds to a direction departing from the skin when the wearable device is bonded to skin of the subject.

14. A wearable device configured to acquire living body information of a subject, the wearable device comprising:
a first layer that has a water-absorbing property;
a second layer that has a waterproof property and is disposed in a first direction relative to the first layer;
a conductive gel part that is disposed in the first direction relative to the second layer and is capable of contacting skin of the subject;
an electrode part that is disposed in the first direction relative to the conductive gel part and includes an electrode electrically connected to the conductive gel part;
a third layer that is flexible and disposed in the first direction relative to the electrode part and includes a conductor electrically connected to the electrode part; and
a fourth layer that is disposed in the first direction relative to the third layer and includes an electronic component electrically connected through the conductor.

15. A detection method, comprising:
detecting an anomaly of the subject based on the living body information acquired by using the wearable device according to claim 1.

16. The detection method according to claim 15, wherein the detection method detects an anomaly of the subject based on the living body information for a detection duration of 72 hours or longer.
